(19) United States
(12) Patent Application Publication
(10) Pub. No.: US 2007/0140991 A1
(43) Pub. Date: Jun. 21, 2007

(54) Pressure sensitive adhesives for cosmetic applications

(76) Inventors: Prithwiraj Maitra, Morristown, NJ (US); William E. McNamara, Middletown, NY (US); John C. Brahms, Morris Plains, NJ (US); Derrick B. McKie, Brooklyn, NY (US); Tao Zheng, Nanuet, NY (US)

Correspondence Address: AVON PRODUCTS, INC., AVON PLACE, SUFFERN, NY, 10901, US

(21) Appl. No.: 11/314,394

(22) Filed: Dec. 21, 2005

(51) Int. Cl. C09D 5/02; A61K 8/72; A61K 8/81

(52) U.S. Cl. 424/53; 524/556; 424/59; 424/405; 424/70.11

(57) ABSTRACT

Compositions and methods are provided for forming a film on a biological surface which are long-lasting, flexible, transfer-resistant, water-proof, and comfortable to the wearer. The compositions generally comprise pressure sensitive adhesive polymers, optionally with the addition of plasticizers, tackifiers, or other film formers, and are useful for formulating cosmetics and personal care products.

### FIELD OF INVENTION

The present invention relates generally to the use of pressure sensitive adhesive polymers in cosmetic products. More particularly, the present invention relates to cosmetic compositions, which are long-lasting, non-transferable, and comfortable to wear.

### BACKGROUND OF THE INVENTION

Pressure sensitive adhesive polymers are a class of materials which adhere to a wide range of surfaces upon application of light pressure. This class of adhesives does not undergo chemical reaction during the bonding process, in contrast to conventional adhesives such as epoxies, but rather adheres to surfaces primarily through Van der Waals forces, and obtains its unique adhesive properties simply from the hysteresis of the thermodynamic work of adhesion.

The range of pressure sensitive adhesive polymers is quite large. Typically, pressure sensitive adhesive polymers are based on polymers derived from acrylics, styrenic block copolymers, or natural rubber. Acrylic pressure sensitive adhesive polymers are generally random copolymers of a long side-chain acrylic (e.g., n-butyl acrylate or 2-ethylhexyl acrylate) with a low glass transition temperature, short side-chain acrylic (e.g., methyl acrylate), and acrylic acid. Pressure sensitive adhesive polymers based on styrenic block copolymers are generally blends of styrene-isoprene-styrene and styrene-isoprene diblocks.

Traditionally, pressure sensitive adhesive polymers have been used as adhesive coatings on non-tacky substrates, or backing layers, to provide tape and the like or to reversibly adhere two surfaces. For example, a non-tacky backing layer, such as a paper or plastic film, may be coated with pressure sensitive adhesive polymers in order to adhere the backing layer to a surface, such as skin. Such adhesives require only slight pressure to be activated or to adhere to a surface. These materials are applied dry to the surface by application of pressure under ambient conditions. Typical examples of pressure sensitive adhesives that utilize such a backing layer are described in the following literature:

U.S. Pat. No. 6,290,659 discloses a method for exfoliating the skin using a flexible exfoliant sheet with a layer of pressure sensitive adhesive.

U.S. Pat. No. 6,713,552 provides aqueous emulsion polymers for use when dry as pressure sensitive adhesives to be applied to a backing layer. Tackifiers, pigments or coloring agents may be added to the adhesive compositions.

U.S. Pat. No. 5,562,112 provides lipstick samplers that can comprise pressure sensitive adhesive stocks with removable liners. It is suggested that the adhesive can be a self-cross linking acrylic. Protective sheets with pressure sensitive adhesives are used to protect lipstick samples from contamination. The pressure sensitive adhesives are not part of a cosmetic formulation.

U.S. Pat. No. 6,342,561 provides organic particulate-filled adhesives comprising ionomeric particulates dispersed in a polymer matrix, such as a styrene-acrylic copolymer, for forming adhesive strips that are used for the removal of unwanted matter on the skin, such as comedomes, hair, and dirt. The backing layer is typically made of woven fabrics and non-woven materials. No cosmetic formulations are provided.

U.S. Pat. No. 6,458,379 discloses whitening cosmetic compositions comprising a sheet-like substrate and an adhesive layer comprising a wet pressure sensitive adhesive. The pressure sensitive adhesive may comprise a water-soluble polymer and a wetting agent.

U.S. Pat. App. No. 2004/0009202 discloses a matrix patch for controlled delivery of cosmetic skincare additives to the skin comprising an insoluble pressure sensitive adhesive matrix that is free of tackifier resins.

U.S. Pat. No. 4,860,774 discloses a fingernail covering comprising a fabric with a layer of pressure sensitive adhesive which may also include plasticizers.

In contrast to the foregoing patents and patent applications, the present invention does not involve the use of pressure sensitive adhesives on a backing layer.

In the medical industry, pressure sensitive adhesive polymers are currently in use for the delivery of drugs or other active ingredients to the skin, for use in wound care dressings, and for adhering medical devices to the skin. For example, U.S. Pat. No. 5,876,855 provides pressure sensitive adhesive compositions comprising acrylic pressure sensitive adhesives and tackifying resins. The compositions can also comprise pigments. Suggested uses include medical applications.

The following patents generally disclose various pressure sensitive adhesive formulations.

U.S. Pat. No. 6,455,161 provides polymer films functioning as barriers to odors that can comprise acrylic polymeric materials.

U.S. Pat. No. 6,846,867 discloses aqueous coating compositions with improved adhesion to friable surfaces comprising emulsion polymers. The emulsion polymers can include acrylates and methacrylates such as butyl methacrylate, methyl acrylate, and hydroxyethyl methacrylate.

U.S. Pat. No. 6,319,992 discloses pressure sensitive adhesives comprising copolymers of butyl acrylate and methacrylic acid for adhesion to various surfaces at room temperature.

U.S. Pat. No. 6,657,011 discloses adhesive compositions comprising adhesive polymers including acrylic acid and methacrylic acid, and, in some embodiments, tackifiers.

U.S. Pat. No. 5,435,879 discloses methods for adhering to a substrate, including adhesives comprising pressure sensitive adhesive polymers along with monomers to increase cohesive strength. The adhesives may also contain tackifiers. Examples include acrylate polymers including butyl acrylate and methacrylic acid. U.S. Pat. No. 6,890,983 discloses compositions and processes for preparing compositions comprising pigment particles and polymer particles that result in coatings with high gloss and solvent resistance. The compositions can comprise acrylic acid and methacrylic acid.

U.S. Pat. No. 6,887,960 discloses bioadhesive compositions comprising copolymer particles of polymerized units from 60 to 99 weight % weight ethylenically unsaturated nonionic monomers, and from 0 to 5 weight % ethylenically unsaturated carboxylic acid monomer, ethylenically unsaturated inorganic acid monomers, or combinations thereof. The disclosed bioadhesives are stated to be useful as matrices for the delivery of therapeutic agents including pharmaceutical, cosmetic, and prophylactic substances. Cosmetic formulations comprising pressure sensitive adhesive polymers are not disclosed.

None of the foregoing patents, however, disclose cosmetic formulations comprising pressure sensitive adhesive polymers.

U.S. Pat. Nos. 5,451,610 and 5,330,747 disclose color cosmetics. containing a silicone-based pressure sensitive adhesive and a pigment. The present invention does not solely employ a silicone pressure sensitive adhesive.

U.S. Pat. No. 6,743,880 provides methods for the preparation of gel-free, hydrophylic copolymers of 2-hydroxyethyl methacrylate and 4-hydroxybutyl acrylate. The copolymers are suggested to be useful in cosmetic applications and as pressure sensitive adhesives. No specific cosmetic formulations are provided. Further, the present invention does not include copolymers of 2-hydroxyethyl methacrylate and 4-hydroxybutyl acrylate.

Currently there is a need for cosmetic products that are long-lasting and non-transferable as well as comfortable for consumers over long periods of wear time. Presently, cosmetic products on the market deliver one or two of the above-mentioned characteristics, but fail to provide all three. Although many approaches have been used in the art, including a variety of synthetic as well as natural polymers, they consistently fail to provide products that are, for example, not tight or uncomfortable on the lips.

It is therefore an object of the present invention to provide color cosmetic compositions comprising pressure sensitive adhesive polymers that are long-lasting, non-transferable, and comfortable, which can be applied to biological surfaces.

### SUMMARY OF THE INVENTION

Surprisingly, it has been found that pressure sensitive adhesive polymers can be utilized in a wet form that can be applied directly to the skin or other biological surface without a backing layer. The. pressure sensitive adhesive polymers of the invention, alone or in combination with other film formers, tackifiers, or plasticizers, can be used to deliver and hold coloring agents on the surface. The resulting films are uniformly colored, glossy, non-tacky and flexible.

One aspect of the invention provides methods for forming films on a biological surface. The biological surface may be any surface to which cosmetics or personal care products are typically applied, including without limitation skin, lips, keratin fibers (hair, eyelashes, and eyebrows), nails and the like. Generally, the method comprises contacting a biological surface with a composition comprising a pressure sensitive adhesive polymer and, optionally, a component selected from the group consisting of plasticizers, tackifiers, film formers or combinations thereof. The plasticizers, tackifiers, and film formers are present in sufficient quantity to bring the glass transition temperature of the composition to between about 5° C. and about 60° C. The resulting film is water resistant, oil resistant, non-tacky and durable. It will typically have a peak tack force less than 4 N.

The inventive films have a glass transition temperature that is within ±30° C. of body temperature. In some cases, the pressure sensitive adhesive polymers will inherently possess a glass transition temperature within this range. In other cases, one or more plasticizers, tackifiers, film formers, or combinations thereof may be required in sufficient quantity to bring the glass transition temperature within ±30° C. of body temperature. By “in sufficient quantity” it is meant that the amount of the components selected from the group consisting of plasticizers, tackifiers, film formers, or combinations thereof will be chosen to achieve a glass transition temperature of the final film-forming composition of between about 5° C. and about 60° C. However, it will be understood that “sufficient quantity” may be 0 (zero) in the case where the pressure sensitive adhesive alone provides a film having a glass transition temperature between about 5° C. and about 60° C.

Another aspect of the invention provides methods for entrapping a functional agent within a film disposed on a biological surface. The functional agent may be any agent which is desired to be held in contact with the surface, including without limitation pigments, pharmaceuticals, cosmeceuticals and the like. The method comprises contacting a biological surface with a functional agent, a pressure sensitive adhesive polymer, and, optionally, one or more plasticizers, tackifiers, film formers, or combinations thereof, to form a water-resistant and durable film. The film typically has a peak tack force less than 4 N and prevents migration of the functional agent from the biological surface. The plasticizers, tackifiers and film formers are present in sufficient quantity (including their absence) to bring the glass transition temperature of the film to between about 5° C. and about 60° C.

Yet another aspect of the invention provides a film adhered to a biological surface. The film according to this aspect of the invention comprises pressure sensitive adhesive polymers. The film has a glass transition temperature between about 5° C. and about 60° C. and a peak tack force less than about 4 N.

An additional aspect of the invention provides cosmetic compositions comprising pressure sensitive adhesive polymers and, optionally, one or more plasticizers, tackifiers, film formers, or combinations thereof. The quantities of plasticizers, tackifiers, film formers, or combinations thereof are present in sufficient quantities to bring the glass transition temperature of the composition to between about 5° C. and about 60° C. The composition furthers comprise one or more functional agents, particularly coloring agents.

These and other aspects of the invention will be better understood by reference to the following detailed description and appended claims.

### DETAILED DESCRIPTION OF THE INVENTION

It is to be understood that the terms used herein have their ordinary and accustomed meanings in the art, unless otherwise specified.

Generally, the present invention provides cosmetic compositions that deliver uniform color to the body such as to the skin, nails, or keratin fibers (hair, eyelashes, and eyebrows) and that are durable (i.e., long-lasting), flexible, transfer-resistant, oil resistant, water-resistant, and comfortable to the wearer. The invention also provides methods for forming films on a biological surface.

The present invention is premised on the discovery that pressure sensitive adhesive polymers, when formulated to provide compositions having glass transition temperatures comparable to body temperature, and peel adhesion strength comparable to the cohesive energy of human skin, can provide cosmetic compositions which obtain heretofore unachievable levels of transfer resistance, flexibility, and comfort.

It is believed that the films of the present invention exhibit superior particle coalescence, resulting in superior mechanical performance in comparison to films of the prior art. Coalescence generally refers to the disappearance of the boundary between two particles in contact, or between one of these and a bulk phase followed by changes of shape leading to a reduction of the total surface area. The minimum film forming temperature (MFFT) denotes the minimum temperature at which a dispersion of pressure sensitive adhesive colloidal particles will coalesce when placed on a substrate as a thin film. The driving force for coalescence is the reduction of the surface energy associated with the particle surface.

One aspect of the invention provides films with a MFFT that is above the glass transition temperature of the aforementioned pressure sensitive adhesive polymers. When the MFFT is above the glass transition temperature, solvent evaporation and particle coalescence occur simultaneously with the solvent evaporation being the rate-limiting step. Therefore, in some embodiments of the invention, the films have sufficient particle mobility to move close to one another and coalesce, thus forming densely packed particle arrays before solvent evaporation is complete. This coalescence gives rise to particle deformation, resulting in a strong, stable linkage of particles, thus yielding films that are mechanically tough due to enhanced intermolecular interactions between particles. Conversely, when the MFFT is around or below the glass transition temperature of the pressure sensitive adhesive polymer, the particle coalescence becomes the rate-limiting step. Therefore the particle deformation must occur after the solvent evaporation. In this case there is reduced particle mobility resulting in little coalescence which leads to little or no particle deformation. As a result, intermolecular interactions are reduced, and thus the mechanical properties of the films are greatly affected.

In some embodiments of the invention, the MFFT is between about 5° C. and about 70° C. Preferably, the MFFT is between about 10° C. and about 40° C. Most preferably the MFFT is between about 15° C. and about 35° C.

One embodiment of the invention is a cosmetic composition comprising a pressure sensitive adhesive polymer; one or more components selected from the group consisting of plasticizers, tackifiers, film formers; or combinations thereof; and one or more coloring agents. Suitable materials for each of these components are provided below.

Pressure Sensitive Adhesive Polymers

Pressure sensitive adhesive polymers useful in the practice of the invention can be based on several types of systems, including but not limited to copolymers of acrylate and methacrylate pressure sensitive adhesives, rubber-based pressure sensitive adhesives, and styrene copolymers (such as styrene-isoprene-styrene (SIS) and styrene-butad iene-styrene (SBS) copolymers).

Non-limiting examples of pressure sensitive adhesive polymers include those based on rubber, such as natural rubber (poly(cis-1,4-isoprene)), methyl methacrylate-isoprene graft copolymers, styrene-butadiene copolymer, butyl rubber, acrylonitrile-butadiene rubber, styrene-isoprene block copolymer, polybutadiene, ethylene-butylene block copolymer, and polychloroprene.

One interesting class of pressure sensitive adhesives includes those based on polar acrylic polymers. For example, these can include statistical or block copolymers based on acrylic acid, alkyl acrylates, and alkyl methacrylates can be used, as well as copolymers of these acrylics with ethylene and vinyl acetate. Also suitable are urethane polymers, polyurethane, chloroprene, polybutadiene, isoprene, neoprene, and the like.

Preferred pressure sensitive adhesive polymers according to one embodiment of the invention are acrylic pressure sensitive adhesives. Specifically preferred are copolymers of butyl acrylate, butyl methacrylate, and acrylic acid. These copolymers are commercially available, for example, under the name Roderm 560 (Rohm and Haas). Another preferred pressure sensitive adhesive according to the invention comprises poly(2-ethylhexylacrylate), which is, for example, commercially available under the name Gel-Tac 100G (Advanced Polymer International) as a 40% solids aqueous dispersion of 15 micron tacky acrylic microspheres.

Useful acrylic copolymers according to the invention are commercially available under the trade names Eastarez 2010, 2020, and 2050 (Eastman Chemical Co.); Acronal V210 (BASF); Mowilith LDM 7255 and Revacryl 491 (Clariant); Flexbond 165 (Air Products).

Specific useful rubber polymeric materials (isoprene and butadiene polymers) according to the invention are commercially available under the trade names Ricon 130 polybutadiene (Atofina Sartomer) and Isolene 40 polyisoprene (Elementis).

Useful polyurethane materials of the invention are commercially available under the trade names Sancure 2104 (Noveon) and Vylon UR 1400 (Toyobo Vylon).

Useful vinyl acetate copolymers according to the invention are commercially available under the trade names PVP/VA S-630 (International Specialty Products) and Flexbond 149 (Air Products).

Useful vinyl alcohol/vinyl acetate copolymers of the invention are commercially available under the trade names Celvol 107 (Celanese) and Elvanol 50-42 (DuPont).

Of course, the foregoing commercially available pressure sensitive adhesive polymers are merely representative of the numerous available polymers known in the art.

It will be understood that the pressure sensitive adhesive polymers of the invention do not include copolymers of 2-hydroxyethyl methacrylate and 4-hydroxybutyl acrylate as the sole pressure sensitive adhesive component. Further, the invention does not embrace the use of silicone-based pressure sensitive adhesive polymers as the sole pressure sensitive adhesive component.

Plasticizers, Tackifiers, and Film Formers

Plasticizers, tackifiers, and film formers can be added to adhesive compositions in order to modify the properties of the adhesive including, for example, the visoelastic behavior of the finished adhesive, such as workability and flexibility. It is well within the skill in the art to select the identity and quantity of such components to vary the physical properties of a film. The blend of these materials with pressure sensitive adhesive polymers can achieve any desired balance of tack, adhesion, and cohesive strength of the film on the biological surfaces such as skin, keratin fibers (hair, eyelashes, and eyebrows), or nails.

Plasticizers are additives that soften a material by either softening the final product or to increase the workability of the material before it hardens. Generally, plasticizers work by embedding themselves between the chains of polymers, spacing them apart or increasing the free volume, and thus lowering the glass transition temperature which makes the material more pliant. Selection of plasticizer is typically based on the solubility parameter of the plasticizer. The solubility parameter of the plasticizers useful in the present invention is typically within about 1.0 (cal/cc)^{1/2}, preferably within about 0.5 (cal/cc)^{1/2} and more preferably within about 0.2 (cal/cc)^{1/2} of that of the pressure sensitive adhesive polymer. In addition, the selection of plasticizer typically takes into account the molecular weights of the plasticizer.

Plasticizers useful in the present invention include, but are not limited to polyethelyne glycol, polypropylene glycol, esters of polyethylene glycol and polypropylene glycol, glycerin, polypropylene glycol-polyethylene oxide copolymer, block copolymers of ethylene oxide and propylene oxide, polyethylene oxide alkylphenyl esters, sorbitol, mannitol, lanolin, and lecithin. Any other plasticizer which meets the foregoing criteria is also contemplated to be useful.

Tackifier resins are typically added to modify the viscoelastic properties of pressure sensitive adhesives. A tackifier can modify properties such as tack; (the ability to stick with finger pressure), adhesion (the binding force of the adhesive), and cohesive strength of a pressure sensitive adhesive. The proper tackifier can lend the adhesive composition sufficient viscous flow properties to bring the adhesive and substrate into intimate contact, thus allowing adhesive bonds to form.

In selecting a suitable tackifier resin, the skilled artisan will be guided by the observation that the solubility parameter of the tackifier will be within about 1.0 (cal/cc)^{1/2}, preferably within about 0.5 (cal/cc)^{1/2} and more preferably within about 0.2 (cal/cc)^{1/2} of that of the pressure sensitive adhesive polymer. In addition, the selection of tackifier is typically based on the softening point of the tackifier. The softening point of the tackifier resins useful in the present invention are typically between about 10° C. and about 150° C. Preferably, the softening point of the tackifier resin is between about 20° C. and about 140° C. and more preferably between about 30° C. and about 130° C.

Suitable tackifiers of the present invention include, but are not limited to any compatible resins, based on solubility parameters described above, or mixtures thereof, including without limitation (1) natural or modified rosins such, for example, as gum rosin, wood rosin, tall-oil rosin, distilled rosin, hydrogenated rosin, dimerized rosin, and polymerized rosin; (2) glycerol and pentaerythritol esters of natural or modified rosins, such, for example as the glycerol ester of pale, wood rosin, the glycerol ester of hydrogenated rosin, the glycerol ester of polymerized rosin, the pentaerythritol ester of hydrogenated rosin, and the phenolic-modified pentaerythritol ester of rosin; (3) copolymers and terpolymers of natural terpenes, e.g., styrene/terpene and alpha methyl styrene/terpene; (4) polyterpene resins having a softening point, as determined by ASTM method E28,58T, of from about 10° to 150° C.; the latter polyterpene resins generally resulting from the polymerization of terpene hydrocarbons, such as the bicyclic monoterpene known as pinene, in the presence of Friedel-Crafts catalysts at moderately low temperatures; also included are the hydrogenated polyterpene resins; (5) phenolic modified terpene resins and hydrogenated derivatives thereof, for example, as the resin product resulting from the condensation, in an acidic medium, of a bicyclic terpene and phenol; (6) aliphatic petroleum hydrocarbon resins having a Ball and Ring softening point of from about 10° to 135° C.; the latter resins resulting from the polymerization of monomers consisting of primarily of olefins and diolefins; also included are the hydrogenated aliphatic petroleum hydrocarbon resins; (7) alicyclic petroleum hydrocarbon resins and the hydrogenated derivatives thereof; and (8) aliphatic/aromatic or cycloaliphaticlaromatic copolymers and their hydrogenated derivatives.

The preferred tackifiers according to the invention include water-based tackifier resins based on rosin esters or terpenes or phenolics.

The film formers of the present invention can include, but are not limited to, film-forming polymers, for example, polyurethane dispersions, acrylic, vinylic or styrene-acrylic polymer dispersions or any other aqueous dispersion of polymers or copolymers.

In some embodiments of the invention, the pressure sensitive adhesive itself can act as a film former, thus obviating the need for a secondary film former. In some embodiments however, the secondary film former can be added with the proviso that the pressure sensitive adhesive requires glass transition temperature adjustment to meet the criteria described herein.

Useful film formers in the practice of the present invention include acrylate film formers that are commercially available, under the names of Daitosol (Kobo Products, Inc,) which is an ethyl methacrylate, n-butyl acrylate, and 2-methylhexyl, acrylate copolymer; Covacryl E14 (Sensient Technologies), an acrylic, long-lasting film; Dermacryl 79 (National Starch & Chemical Company), an acrylate/octylacrylamide copolymer; and Allianz LT-120 (International Specialty Products) which comprises acrylate, C-1-2 succinates, and hydroxyacrylate copolymers. Other useful film formers can be found in the CTFA Handbook of Cosmetic Ingredients

Coloring Agents

Where the product is a color cosmetic for the body, such as a lipstick, lip gloss, nail enamel, mascara, foundation, and the like, the compositions will further comprise one or more coloring agents. It is within the skill in the art to choose coloring agents and combinations of coloring agents to produce a desired color. Suitable coloring agents, including pigments, lakes, and dyes, are well known in the art and are disclosed in the C.T.F.A. Cosmetic Ingredient Handbook, First Edition, 1988, the contents or which are hereby incorporated by reference. Organic pigments include, for example, FD&C dyes, D&C dyes, including D&C Red, Nos. 2, 5, 6, 7, 10, 11, 12, 13, 30 and 34, D&C Yellow No. 5, Blue No. 1, Violet No. 2. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, y-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Other suitable coloring agents include ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like. The coloring agents may be surface modified, with, for example, fluoropolymers, to adjust one or more characteristics of the coloring agent as described in, for example, U.S. Pat. Nos. 6,471,950, 5,482,547, and 4,832,944, the contents of which are hereby incorporated by reference. Fluoropolymers may be incorporated into the present invention as a coating on pigment particles that at least partially covers the surface of the pigment particles. Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference.

In contrast to the conventional use of pressure sensitive adhesive materials which has included the application of a pressure sensitive adhesive in a dry form onto a backing layer to facilitate easy handling and application to the skin or other surface with one layer being non-tacky, the present invention uses compositions comprising pressure sensitive adhesive polymers in wet form that are applied directly to the skin, keratin fibers (hair, eyelashes, and eyebrows), nails, and the like without a backing layer to result in a uniformly colored, glossy, and non-tacky film.

Because the present compositions allow for the use of tackifiers, plasticizers, and/or film formers to modify glass transition temperature, the range of the glass transition temperature of the pressure sensitive adhesive polymers of the invention is from about −120° C. to to about 40° C. In choosing the amount of pressure sensitive adhesive polymers, plasticizers, tackifiers, film formers, or combinations thereof, the skilled artisan will be guided by the requirement that the glass transition temperature of the final film is between about 5° C. and about about 60° C. In addition, the final film is brought to the condition of having a peak tack force of less than about 4 N. More preferably, the glass transition temperature of the final film is between about 10 and about 50° C., the peak tack force of the final film is between about 0.05 and about 2 N. Above the glass transition temperature, polymers can undergo plastic deformation. Peak tack force measures the final tack property of a composition, and is obtained with a tensile measurement instrument equipped with a 5 Kg load cell such as Instron or Texture Analyzer equipment.

The preferred pressure sensitive adhesive polymers of the present invention may be characterized by one or more physical properties including peel adhesion strength and loop tack.

In one embodiment of the invention, the pressure sensitive adhesive polymers have a peel adhesion strength less than about 30 N/10 mm. Preferably, the peel adhesion strength of the pressure sensitive adhesive polymers is between about 0.5 and about 19 N/10 mm. Peel adhesion is determined in accordance with ASTM D-3330-78, PSTC-101 and is a measure of the force required to remove a coated, flexible sheet material from a test panel at a specific angle and rate of removal. A one-inch width of a coated sheet is applied to a horizontal surface of a clean, stainless steel test plate with at least five lineal inches of the coated sheet material in firm contact with the steel plate. A hard rubber roller is used to firmly apply the strip and remove all discontinuities and entrapped air. The free end of the coated strip is then doubled back nearly touching itself so that the angle of removal of the strip from the steel plate will be 180 degrees. The free end of the test strip (the one pulled) is attached to the adhesion tester scale (an Instron tensile tester or Harvey tensile tester). The test plate is then clamped in the jaws of the tensile testing machine capable of moving the plate away from the scale at a constant rate of 12 inches per minute. The scale reading in pounds is recorded as the tape is peeled from the steel surface.

In a further embodiment of the invention, the pressure sensitive adhesive polymers have a loop tack of less than about 40 N. Preferably, the loop tack of the pressure sensitive adhesive polymers is between about 1 and about 20 N. Loop tack is a measure of the force required to remove a standard adhesive coated Mylar film loop from a standard (PSTC) stainless steel plate at 73° F. after only nominal contact of the test strip with the steel plate in the absence of significant pressure. A one-half by four-inch strip of one mil Mylar film coated with the sample adhesive is formed into a loop with the adhesive side out, and the loop is applied to a stainless steel plate until the tape loop contacts 0.5 square inch of surface area on the plate. The loop is retracted from the plate at a rate of 12 inches per minute, and loop tack is defined as the force observed when the final portion of the adhesive strip separates from the test plate. Specifically, the steel test plate is inserted in the lower jaws of an Instron tensile tester while the upper portion of the loop is clamped in the upper jaw of the tester and is moved downward toward the test plate at a rate of 12 inches per minute. When the test loop has contacted 0.5 square inch of test plate area, the direction of travel of the upper jaw of the Instron tester is reversed and set to remove the loop from the plate at a rate of 12 lineal inches per minute.

Optionally, the composition may comprise one or more fluoropolymers in order to enhance water and oil resistance. Suitable fluoropqlymers include, but are not limited to polymers derived from at least one fluorine containing monomer, but may also incorporate monomers that do not contain fluorine or other halogens. The fluoropolymer preferably contains at least 25 weight percent fluorine. Examples of fluoropolymers include but are not limited to polytetrafluoroethylene (PTFE), polychlorotrifluoroethylene (PCTFE), perfluoroalkoxy (PFA), fluorinated ethylene propylene (FEP), polyvinylidene fluoride (PVDF), ethylenetetrafluoroethylene (ETFE), ethylenechlorotrifluoroethylene (ECTFE) and mixtures thereof. A preferred average particle size of the fluoropolymers ranges from 0.05 to 5 microns.

The compositions of the invention may optionally comprise other active and inactive ingredients, including, but not limited to, excipients, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, coloring agents, staining agents, depigmenting agents, hypopigmenting agents, preservatives, stabilizers, pharmaceutical agents, photostabilizing agents, and mixtures thereof. In addition to the foregoing, the personal care products of the invention may contain any other compound for the treatment of skin disorders.

Another embodiment of the invention provides methods comprising contacting a biological surface with a composition as described above.

In yet another embodiment of the invention is provided a method for forming a film on a biological surface herein. Further, methods are provided for entrapping a functional agent within an elastomeric film disposed in a biological surface comprising a composition as described above. Functional agents suitable in the present invention include, but are not limited to coloring agents, such as pigments, lakes, and dyes; opacifiers, pearling agents, insect repellants, UV absorbers, UV blockers, antiperspirants, anti-acne agents, moisturizers, conditioners, and tooth whiteners.

The cosmetic formulations of some embodiments of the invention may also comprise a cosmetically acceptable carrier or vehicle acceptable for topical application to the skin, keratin fibers (hair, eyelashes, and eyebrows), or nails. The pressure sensitive adhesive polymers may be soluble in the carrier or may be suspended as a colloidal dispersion in the vehicle.

Examples of such vehicles include, but are not limited to, water and aqueous systems; glycerin; various hydrophilic solvents including alcohols such as ethanol, methanol, propyl and other alcohols; or any combinations thereof. Hydrocarbon solvents useful in the practice of the invention, in some embodiments, may be chosen from mineral oils, mineral solvents, mineral spirits, petroleum, waxes, synthetic hydrocarbons, animal oils, vegetable oils, and mixtures of various hydrocarbons. In some embodiments, isododecane or a light paraffinic solvent are used. In other embodiments, a non-hydrocarbon solvent such as amyl acetate, butyl acetate, isobutyl acetate, ethyl acetate, propyl acetate or isopropyl acetate are used.

In addition, the vehicle of the compositions according to the present invention can be in the form of a suspension, gel, or solution, formulation. Other suitable topical carriers include an anhydrous liquid solvent such as oil and mono- or polyhydric alcohols; aqueous-based single phase liquid solvent (e.g. hydro-alcoholic solvent system); anhydrous solid and semisolid (such as a gel and a stick); and aqueous based gel and mousse system.

The product form of the present invention may be, for example, an aerosol, cream, emulsion, gel, liquid, lotion, mousse, patch, pomade, powder, solid, spray, stick or towelette.

The compositions of the invention are contemplated to be useful in any number of commercial products, including but not limited to oral care products, surfactant/cleaning products, over-the-counter drugs and pharmaceuticals, artificial tanning products, sporting camouflage, foot-care products, liquid and bar soaps, anti-perspirant and deodorant products, fragrance-emitting products, analgesics, insect repellents, poison ivy products including poison ivy blocks, jellyfish protectants, hair care products, shampoos, conditioners, hair colors, hair styling products, hair mascaras, and decorative cosmetics including various make-up products, pressed powder, moisturizers, facial treatment products such as foundations and concealers, mascara, eye liners, body art, lipsticks, lip gloss, lip balms and nail care products.

The comsetic compositions of the invention will typically comprise between about 0.01% and about 90.0% by weight of pressure sensitive adhesive polymers and between about 1.0 and about 99.0% by weight of carrier.

In a preferred embodiments, the cosmetic compositions of the invention will typically comprise between about 0.5% and about 35.0% by weight of pressure sensitive adhesive polymers and between about 5.0 and about 90.0% by weight of carrier.

### EXAMPLE 1

Lip gloss prototypes using pressure sensitive adhesives are provided. Table 1 provides prototype formulations using Roderm as a film former, followed by the addition of pressure sensitive adhesives (Roderms).

TABLE 1

| | Sample | Sample | Sample | Sample | Sample |
|---|---|---|---|---|---|
| | 1 | 2 | 3 | 4 | 5 |
| Pigment dispersion^{a} | 0 | 40 | 0 | 0 | 0 |
| Water dispersible | 0 | 0 | 0 | 0 | 10 |
| Pigments (Kobo) | | | | | |
| Pigment/Fluorosil | 10 | 0 | 10 | 15 | 0 |
| dispersion^{b} | | | | | |
| Pressure Sensitive | 20.6 | 20 | 27.8 | 12 | 19 |
| Adhesive (Roderm | | | | | |
| 560 ™, Rohm & | | | | | |
| Haas) | | | | | |
| Film former | 38 | 8 | 31 | 16 | 0 |
| (Daitosol 5000 ™, | | | | | |
| Kobo) | | | | | |
| Water | 31 | 31.8 | 30.8 | 56.5 | 30.6 |
| Rheology modifier | 0 | 0 | 0 | 0 | 0.4 |
| (Xanthan gum) | | | | | |
| Film former | 0 | 0 | 0 | 0 | 40 |
| (Covacryl E14) | | | | | |
| Rheology modifier | 0.4 | 0.2 | 0.4 | 0.5 | 0 |
| (TIC Gum CMC) | | | | | |
| Total | 100 | 100 | 100 | 100 | 100 |

^{a}water based pigment dispersion containing Daitosol (Kobo Products, Inc.)
^{b}Pigment grind prepared using (organic/inorganic) fluorosilicone-treated pigments (80-wt %) with a water soluble fluorosilicone (Fluorosil 2110) as grind media (20 wt %).

The formulations in Table 1 were prepared by first adding CMC/Xanthan gum in water at 50° C. and mixing for 20 minutes using an overhead mixer until a homogenous gel structure was formed. This was followed by the addition of pigment to the aqueous gel structure until a homogenous dispersion was obtained. This was then slowly cooled to room temperature. Next, Daitosol 5000 and Covacryl E14 were added at room temperature and mixed for another 15 minutes. This was followed by the addition of Roderm 560, and then by mixing for another 15 minutes.

### EXAMPLE 2

The transfer resistance of the lip gloss formulations of Example 1 was examined in comparison to the commercial lip coloring products Lipfinity™ (Procter & Gamble) and Lip Polish™ (Maybelline) using a modification of the transfer resistance testing protocol of U.S. Pat. No. 6,074,654, the disclosure of which is hereby incorporated by reference. The testing protocol is described below.

Transfer Resistance Test Method

This method may be utilized to determine the water and oil transfer resistance and adhesion properties of a cosmetic film. This test predicts the ability of a cosmetic film to resist color transfer to objects contacting the skin. Such objects include clothing, handkerchiefs or tissues, napkins and implements such as cups, glasses and table wear, and oily fingers or objects such as oily foods.

Films formed from cosmetic compositions exhibit a degree of transfer resistance directly proportional to the hardness and solvent-resistance of the film. The hardness and solvent-resistance can be expressed as a function of the blot and rub test as described below. Standard safety measure should be observed when performing this test.

Equipment:

(1) Glass plates;
(2) Collagen sausage casing such as Nippi Casing F Grade;
(3) Constant humidity chamber adjusted to 95% relative humidity;
(4) Utility Knife;
(5) Ruler;
(6) Single-sided adhesive tape;
(7) Double-sided adhesive tape;
(8) 25 micron thickness slot draw-down bar;
(9) White Styrofoam dinner plate such as Amoco Selectables™ Plastic DL® Tableware;
(10) 1.5 inch diameter circular metal punch;
(11) 1 kilogram weight;
(12) Vegetable oil;
(13) Brush-tip cosmetic applicator; and
(14) Lint-Free Wiper, such as Kimwipes® EX-L.
   Procedure:
(1) Prepare a 3×4 inch sheet of collagen sausage casing by hydrating it in a 90% relative humidity chamber for at least 24 hours.
(2) Remove the collagen sheet to ambient conditions and immediately wrap tightly around the glass plate. Attach the collagen sheet to the glass using adhesive tape. The collagen surface should be flat and free of wrinkles.
(3) Allow the collagen-wrapped slide to equilibrate at ambient conditions for 24 hours.
(4) Draw down thin (1 mil), uniform films of cosmetic on the collagen.
(5) Allow the cosmetic samples on the collagen surface to sit at ambient conditions for 1 hour.
(6) Using a pipet, drop three drops of vegetable oil onto the right side of the film. Using another pipet, drop three drops of water onto the left side of the film.
(7) Separately for the oil and water sections, distribute the oil and water evenly over the film surface with cosmetic brush applicators, brushing lightly.
(8) Allow the oil and water to remain on the film undisturbed for 15 minutes.
(9) Using a lint-free wiper, carefully blot excess oil and water from the film surface. Apply as little pressure as possible during this step.
(10) Cut two disks from a clean, white Styrofoam dinner plate using a 1.5 inch diameter circular punch. The surface and edges of each disk should be smooth and even.
(11) Firmly attach with double-sided adhesive tape the disks from step (10) to the bottom surface of the 1 kg weight.
(12) Set the weight on top of the cosmetic sample applied to the collagen surface from step (5) above so that disk #1 is in contact with the oil section of the film. And disk #2 is in contact with the water section of the film. It is important to position the weight gently so that excess force beyond 1 kg is not applied.
(13) Grasping the top of the 1 kg weight, carefully rotate the disk through 360 degrees while maintaining the 1 kg force on the film. Do not lift or press the weight into the film during the rotating motion to the weight. The entire 360 degree rotation should be completed within a time interval between 3 and 5 seconds.
(14) Lift the weight straight up off the film surface and carefully remove the disk from the weight avoiding damage to the disk.
(15) Color transfer on individual discs is based on visual assessment of the discs compared to commercial products as positive and negative benchmarks. The positive control used is Lipfinity™ (base coat) while negative control used is the Lip polish™ product.

(16) The criteria used in the “Star Grading System” for measuring the degree of transfer is explained in Table 2.

TABLE 2
Star Grading System

| Visual Assessment of Transfer | Scale |
|---|---|
| Less than Negative Control | * |
| Equal to or slight better than Negative control | ** |
| Between Negative and Positive Control | *** |
| About equal to positive control | **** |
| Better than positive control | ***** |

The results indicate that the lip gloss formulations of Example 1 exhibit superior transfer resistance to both the positive (Lipfinity™ base coat) and negative (Lip polish™) controls. In each case noticeably less pigment had transferred to the Styrofoam disk for both formulations of Example 1 than for the control products. The results are quantified on the basis of the Star Grading System as shown below in Table 4.

The flexibility of the lip gloss formulations of Example 1 were examined using a modification of the flexibility testing protocol described in patent U.S. Pat. No. 6,074,654, the contents of which are hereby incorporated by reference. The flexibility of a cosmetic film is an important to both the durability (long-wear) and comfort properties of a cosmetic film.

Flexibility is measured by the latex stretch test. This test predicts the ability of the color film to resist flaking or peeling types of failure after application by movement of the skin during normal activities. The flexibility latex stretch test is based on the weight-loss measurement before and after the latex stretch.

Equipment:

(1) Ansell Industrial technicians unlined gloves (12″ length, 17 mil) USDA Accepted #390, Size 9
(2) Slanted Eyeshadow Brushes from Avon Products, Inc.
(3) Analytical balance (4 decimal places); and
(4) Ruler.
   Procedure:
(1) Cut a 1 inch wide band from the wrist area of the glove, avoiding the ribbing and thumb.
(2) Mark off a 1×1 inch block in the center of smooth side of the band, avoiding the embossed number.
(3) Weigh and record the weight of the latex band; hereinafter referred to as A.
(4) Determine the initial weight of the cosmetic to be applied to the band in order to produce a dried film weighing 20 mg. This is determined by dividing 20 mg by the weight percent of non-volatile material present in the cosmetic. For example, 40 mg of a cosmetic with 50% non-volatile content must be applied to the band in order to yield a 20 mg dried film.
(5) Using a clean eyeshadow brush, evenly apply the amount of cosmetic determined in step (4) over the 1×1 inch area of the band as marked in step (2).
(6) Immediately weigh and record the combined weight of the latex band and applied cosmetic. The weight of wet film with the latex band is referred to as B.
(7) Allow the sample on the latex band from step (6) to sit at ambient room conditions for 24 hours. The optimum test conditions to reliably correlate this test to the physical characteristics of the composition require that the film be dry. By dry it is meant that at least 90% of the volatile carrier of the cosmetic composition has evaporated.
(8) Weigh and record the combined weight of the latex band A and the applied cosmetic film; hereinafter referred to as C. Subtract A from C to determine the dried film weight D (D=C−A). This weight should be 20±2 mg.
(9) Gently stretch the latex band so that the marked film length changes from 1.00 inches to 1.75 inches.
(10) Upon observing loosened film pieces on the latex band, remove the film pieces from the latex band by vigorously wiping a clean eyeshadow brush across the surface of the film: 10 times wiping in vertical direction and 10 times wiping in horizontal direction.
(11) Carefully allow the latex band to return to its approximate original shape.
(12) Record the weight of the latex band (with the remaining cosmetic); herein referred to as E.

(13) A “Star Grading System” is used based on percentage weight loss (“PWL”) to grade the flexibility of the films as follows:

TABLE 3

| Weight Loss | Scale |
|---|---|
| 100-50% | * |
| 30-50% | ** |
| 15-30% | *** |
| 5-15% | **** |
| 0-5% | ***** |

The percent weight loss of the cosmetic film is calculated using the following equation:
Percent Weight Loss (PWL)=[1−(E−A)/(C−A)]×100%

For some very flexible films, the percentage weight loss may be negligible. Therefore, in some case, due to some dust transferred from the brush, the PWL value may become negative (weight gain).

Steps (1) through (12) are repeated three times for each cosmetic formulation tested. The average of the three PWL values is determined; herein referred to as Average Percent Weight Loss (“APWL”). Low APWL values (i.e., 0-5%) correspond to flexible films having a desirable adhesive and cohesive balance of the film. The flexibility test results for the lip gloss formulations of Example 1 are quantified on the Star Grading System as shown in Table 4.

TABLE 4

| | Transfer | Transfer | |
|---|---|---|---|
| Formula # | (Oil) | (Water) | Flexibility |
| Sample 1 | *** | * | ***** |
| Sample 2 | ***** | * | ***** |
| Sample 3 | **** | * | ***** |
| Sample 4 | **** | * | ***** |
| Sample 5 | ***** | * | ***** |

The invention having been described by the foregoing description of the preferred embodiments, it will be understood that the skilled artisan may make modifications and variations of these embodiments without departing from the spirit or scope of the invention as set forth in the following claims.

1. A method for imparting transfer resistance of a cosmetic to a body comprising:
applying to said body a composition comprising:
(i) a pressure sensitive adhesive polymer; and
(ii) a component selected from the group consisting of plasticizers, tackifiers, or film formers, or combinations thereof, present in sufficient quantity to bring the glass transition temperature of said composition to between about 5° C. and about 60° C.;
wherein said cosmetic forms a film and the resulting film being water-resistant, oil resistant, durable and non tacky, and having a peak tack force of less than about 4 N.

2. The method of claim 1 wherein said pressure sensitive adhesive polymer has a peel adhesion strength less than 30 N/10 mm.

3. The method of claim 1 wherein said pressure sensitive adhesive polymer is selected from the group consisting of copolymers of acrylate and methacrylate pressure sensitive adhesives, rubber-based pressure sensitive adhesives, and styrene copolymers.

4. The method of claim 1 wherein said pressure sensitive adhesive polymer is a styrene copolymer consisting of styrene-isoprene-styrene (SIS) and styrene-butadiene-styrene (SBS) copolymers.

5. The method of claim 1 wherein said pressure sensitive adhesive polymer is a rubber-based polymer selected from the group consisting of natural rubber (poly(cis-1,4-isoprene)), methyl methacrylate-isoprene graft copolymers, styrene-butadiene copolymer, butyl rubber, acrylonitrile-butadiene rubber, styrene-isoprene block copolymer, polybutadiene, ethylene-butylene block copolymer, and polychloroprene.

6. The method of claim 1 wherein said pressure sensitive adhesive polymer is a polar acrylic polymer selected from the group consisting of statistical or block copolymers based on acrylic acid, alkyl acrylates, and alkyl methacrylates, urethane polymers, polyurethane, chloroprene, polybutadiene, isoprene and neoprene.

7. The method of claim 1 wherein said pressure sensitive adhesive polymer is an acrylic pressure sensitive adhesive.

8. The method of claim 7 wherein said pressure sensitive adhesive is a copolymer of butyl acrylate, butyl methacrylate or acrylic acid.

9. The method of claim 7 wherein said pressure sensitive adhesive is Roderm 560.

10. The method of claim 7 wherein said pressure sensitive adhesive is poly(2-ethylhexylacrylate), commercially available as Gel-Tac 100G.

11. The method of claim 1 wherein said pressure sensitive adhesive polymer has a loop tack of about 0.01 to about 40 N/10 mm.

12. A method for forming a film on a body comprising:
contacting said surface with a composition comprising:
(i) a pressure sensitive adhesive polymer; and
(ii) a component selected from the group consisting of plasticizers, tackifiers, or film formers, or combinations thereof, present in sufficient quantity to bring the glass transition temperature of said composition to between about 5° C. and about 60° C.; the resulting film being water-resistant, oil resistant, durable and non tacky, and having a peak tack force of less than about 4 N.

13. A method for entrapping a functional agent within a film disposed on a biological surface comprising: contacting said biological surface with said functional agent, a pressure sensitive adhesive polymer, and a component selected from the group consisting of plasticizers, tackifiers, film formers, and combinations thereof to form a water-resistant durable film thereon, said film having a peak tack force of less than about 4 N; thereby preventing migration of said functional agent from said biological surface; wherein one or more plasticizers or tackifiers are present in sufficient quantity to bring the glass transition temperature of said composition to between about 5° C. and about 60° C.

14. The method of claim 13 wherein said functional agent is selected from the group consisting of pigments, lakes, dyes, opacifiers, pearling agents, insect repellants, UV absorbers, UV blockers, antiperspirants, anti-acne agents, moisturizers, conditioners, and tooth whiteners.

15. A composition for application as a cosmetic to the body, said composition having: a glass transition temperature between about 5° C. and about 60° C. and a peak tack force of less than about 4 N, said film comprising a pressure sensitive adhesive polymer.

16. A cosmetic composition comprising:
(i) a pressure sensitive adhesive polymer, and
(ii) a component selected from the group consisting of plasticizers, tackifiers, film formers, and combinations thereof, present in sufficient quantity to bring the glass transition temperature of said composition to between about 5° C. and about 60° C.; and
(iii) one or more coloring agents;
the resulting composition being water-resistant and durable and having a peak tack force of less than about 4 N.
